(19) United States
(12) Patent Application Publication
(10) Pub. No.: US 2019/0133979 A1
(43) Pub. Date: May 9, 2019

(54) METHOD FOR ENHANCING DELIVERY OF THERAPEUTIC DRUGS TO TREATMENT SITES

(71) Applicant: Chang Gung University, Taoyuan City (TW)

(72) Inventors: Yunn-Hwa MA, Taoyuan City (TW); Yann-Lii LEU, Taoyuan City (TW); Jender WU, Taoyuan City (TW); Kuo-Chen WEI, Taoyuan City (TW); Yi-Ching LU, Taoyuan City (TW)

(73) Assignee: Chang Gung University, Taoyuan City (TW)

(21) Appl. No.: 16/237,760

(22) Filed: Jan. 2, 2019

Related U.S. Application Data
(63) Continuation-in-part of application No. 13/899,338, filed on May 21, 2013.

(51) Int. Cl. A61K 31/192; A61K 41/00; A61K 31/235; A61K 31/166; A61K 31/216; A61K 31/352; A61K 33/24; A61P 35/00; A61N 2/00

(52) U.S. Cl. CPC A61K 31/192; A61K 41/00; A61K 31/235; A61K 31/166; A61N 2/002; A61K 31/352; A61K 33/24; A61P 35/00; A61K 31/216

(57) ABSTRACT

Disclosed herein is a method for enhancing uptake of magnetic nanoparticles (MNPs) having a therapeutic agent associated therein to a target site (e.g., a tumor), thereby resulting in elevated level of therapeutic agents being accumulated in the target site. The method comprises concurrently administering a sufficient amount of a polyphenolic compound and MNPs to the target site. Also disclosed herein is a method for treating a cancer in a subject. The method comprises concurrently administering an effective amount of the polyphenolic compound and MNPs to the subject, so as to ameliorate or alleviate symptoms associated with the cancer.

### CROSS-REFERENCES TO RELATED APPLICATIONS

The present application is a continuation-in-part of U.S. application Ser. No. 13/899,338, filed May 21, 2013, which claims priority to a Taiwan patent application NO:101118169, filed May 22, 2012; the entire contents of which are incorporated herein by reference.

### BACKGROUND OF THE INVENTION

### 1. Field of the Invention

The present disclosure relates to drug delivery. More particularly, the disclosure invention relates to methods for enhancing the delivery of anti-cancer drugs to tumor cells.

### 2. Description of Related Art

Magnetic nanoparticles (MNPs) are one of the most established drug delivery carriers. MNPs have a myriad of potential applications, including drug delivery for targeted therapy, gene delivery, contrast agents in magnetic resonance imaging (MRI) magnetic hyperthermia, biosensors, and etc.

Cellular uptake of MNPs depends on certain characteristics of the particles, including size, shape, functional groups on the surface of the nanoparticles and etc. The superparamagnetic characteristics of MNPs allow MNPs acquiring or losing the magnetic responsiveness rapidly in response to the presence or absence of an external magnetic field. External magnetic force may thus guide MNPs to desired target site, improve accumulation in the target tissue, and enhance cellular uptake of MNPs.

In the present study, inventors unexpectedly found that some polyphenolic compounds may independently enhance cellular uptake of MNPs, resulting in much higher amount of MNPs being accumulated in the cell. Thus, these polyphenolic compounds may act as an adjuvant to assist the delivery of therapeutic components associated therewith and/or encapsulated therein the MNPs to a target site, thereby achieves the purpose of treatment on the target site.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

According to one aspect of the present disclosure, there is provided a method for enhancing the uptake of a magnetic nanoparticle having an anti-cancer drug associated therein to a tumor cell. The method comprises concurrently administering to the tumor cell a sufficient amount of a polyphenolic compound of formula (I), a pharmaceutically acceptable salt, solvate or ester thereof, and the magnetic nanoparticle,

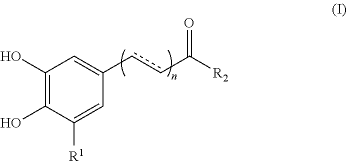

wherein, in the formula (I),

n is 0 or 1;

[symbol] represents a single or double bond;

R₁ is hydrogen or hydroxyl; and

R₂ is hydroxy, —NH₂, or —OR₃, in which R₃ is a heteroaryl optionally substituted with a phenyl having at least two hydroxyl groups substituents.

According to optional embodiments of the present disclosure, the method further comprises the step of administering a magnetic field to the tumor cell.

According to certain embodiments of the present disclosure, in the formula (I), n is 0, and R₁ and R₂ are respectively hydroxyl.

According to certain embodiments of the present disclosure, in the formula (I), n is 0, R₁ is hydroxyl, and R₂ is methoxyl.

According to certain embodiments of the present disclosure, in the formula (I), n is 0, R₁ is hydroxyl, and R₂ is propoxyl.

According to certain embodiments of the present disclosure, in the formula (I), n is 0, R₁ is hydroxyl, and R₂ is —NH₂.

According to certain embodiments of the present disclosure, in the formula (I), n is 0, R₁ is hydrogen, and R₂ is hydroxyl.

According to certain embodiments of the present disclosure, in the formula (I), n is 1, [symbol] is a double bond, R₁ is hydrogen, and R₂ is hydroxyl.

According to certain embodiments of the present disclosure, in the formula (I), n is 1, [symbol] is a single bond, R₁ is hydrogen, and R₂ is hydroxyl.

According to certain embodiments of the present disclosure, in the formula (I), n is 0, R₁ is hydroxyl, and R₂ is

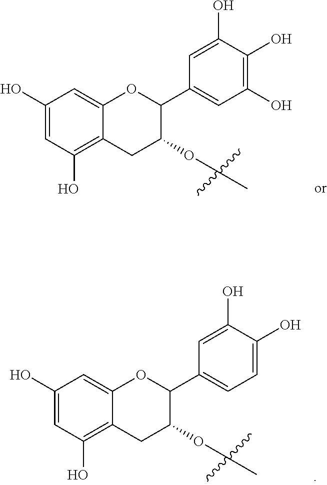 [SMILES: *O[C@@H]1Cc2c(O)cc(O)cc2OC1c1cc(O)c(O)c(O)c1 ; *O[C@@H]1Cc2c(O)cc(O)cc2OC1c1ccc(O)c(O)c1]

In another aspect, the present disclosure is directed to a method for enhancing the uptake of a magnetic nanoparticle having an anti-cancer drug associated therein to a tumor cell. The method comprises concurrently administering to the tumor cell a sufficient amount of a polyphenolic compound and the magnetic nanoparticle, wherein the polyphenolic compound is selected from the group consisting of flavanone, flavone, flavonol, and flavan-3-ol.

According to optional embodiments of the present disclosure, the method further comprises the step of administering a magnetic field to the tumor cell.

Examples of flavanone suitable for use in the present method include, but are not limited to, butin, eruiductyol, and sterubin.

Examples of flavonol suitable for use in the present method include, but are not limited to, quercetin, fisetin, myricetin, phamnetin, luteoforol, epigallocatechin, and epicatechin.

Examples of flavan-3-ol suitable for use in the present method include, but are not limited to, epigallocatechin (EGC), catechin, gallocatechin (GC), and epicatechin

(EC).

Examples of flavone suitable for use in the present method include, but are not limited to, luteolin, 6-hydroxyluteolin, tricetin, hypolaetin, and nepetin.

According to some embodiments of the present disclosure, a sufficient amount of ECG is concurrently administering with the magnetic nanoparticles to the tumor cell.

According to further embodiments of the present disclosure, a sufficient amount of quercetin is concurrently administering with the magnetic nanoparticles to the tumor cell.

In a further aspect, the present disclosure is directed to a method for treating a cancer of a subject. The method comprises concurrently administering to the subject an effective amount of a polyphenolic compound and a magnetic nanoparticle having an anti-cancer drug associated therein, wherein:

the polyphenolic compound is selected from the group consisting of a compound of formula (I), a pharmaceutically acceptable salt, solvate or ester thereof, flavanone, flavone, flavonol, and flanvan-3-ol;

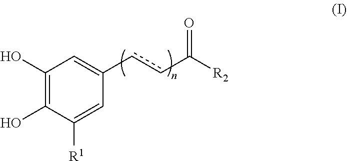

wherein, in the formula (I),

n is 0 or 1;

[symbol] represents a single or double bond;

R₁ is hydrogen or hydroxyl; and

R₂ is hydroxy, —NH₂, or —OR₃, in which R₃ is a heteroaryl optionally substituted with a phenyl having at least two hydroxyl substituents.

According to optional embodiments of the present disclosure, the method further comprises the step of administering a magnetic field to the tumor cell.

According to certain embodiments of the present disclosure, the compound of formula (I) is concurrently administering to the subject with the magnetic nanoparticle, wherein in the formula (I), n is 0, and R₁ and R₂ are respectively hydroxyl.

According to certain embodiments of the present disclosure, the compound of formula (I) is concurrently administering to the subject with the magnetic nanoparticle, wherein in the formula (I), n is 0, R₁ is hydroxyl, and R₂ is methoxyl.

According to certain embodiments of the present disclosure, the compound of formula (I) is concurrently administering to the subject with the magnetic nanoparticle, wherein in the formula (I), n is 0, R₁ is hydroxyl, and R₂ is propoxyl.

According to certain embodiments of the present disclosure, the compound of formula (I) is concurrently administering to the subject with the magnetic nanoparticle, wherein in the formula (I), n is 0, R₁ is hydroxyl, and R₂ is —NH₂.

According to certain embodiments of the present disclosure, the compound of formula (I) is concurrently administering to the subject with the magnetic nanoparticle, wherein in the formula (I), n is 0, R₁ is hydrogen, and R₂ is hydroxyl.

According to certain embodiments of the present disclosure, the compound of formula (I) is concurrently administering to the subject with the magnetic nanoparticle, wherein in the formula (I), n is 1, [symbol] is a double bond, R₁ is hydrogen, and R₂ is hydroxyl.

According to certain embodiments of the present disclosure, the compound of formula (I) is concurrently administering to the subject with the magnetic nanoparticle, wherein in the formula (I), n is 1, [symbol] is a single bond, R₁ is hydrogen, and R₂ is hydroxyl.

According to certain embodiments of the present disclosure, the compound of formula (I) is concurrently administering to the subject with the magnetic nanoparticle, wherein in the formula (I), n is 0, R₁ is hydroxyl, and R₂ is

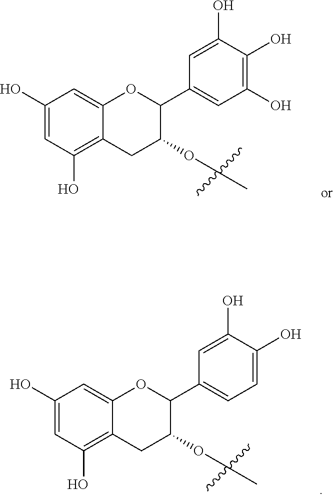 [SMILES: *O[C@@H]1Cc2c(O)cc(O)cc2OC1c1cc(O)c(O)c(O)c1 ; *O[C@@H]1Cc2c(O)cc(O)cc2OC1c1ccc(O)c(O)c1]

Examples of flavanone suitable for use in the present method include, but are not limited to, butin, eruiductyol, and sterubin.

Examples of flavonol suitable for use in the present method include, but are not limited to, quercetin, fisetin, myricetin, phamnetin, luteoforol, epigallocatechin, and epicatechin.

Examples of flavan-3-ol suitable for use in the present method include, but are not limited to, epigallocatechin (EGC), catechin, gallocatechin (GC), and epicatechin (EC).

Examples of flavone suitable for use in the present method include, but are not limited to, luteolin, 6-hydroxyluteolin, tricetin, hypolaetin, and nepetin.

According to certain embodiments of the present disclosure, EGC is concurrently administering to the subject with the magnetic nanoparticle.

According to certain embodiments of the present disclosure, quercetin is concurrently administering to the subject with the magnetic nanoparticle.

According to embodiments of the present disclosure, the cancer may be selected from the group consisting of bladder cancer, bone cancer, bone marrow cancer, brain cancer, breast cancer, cholangiocarcinoma, colon cancer, esophagus cancer, Ewing's sarcoma, gastro-intestine cancer, gum cancer, head cancer, Hodgkin's disease, kidney cancer, liver cancer, lung cancer, larynx cancer, melanoma, multiple myeloma, nasopharynx carcinoma, non-small-cell lung (NSCL) cancer, leukemia, liver cancer, nasopharynx cancer, neck cancer, neuroblastoma, ovary cancer, pancreatic cancer, prostate cancer, rectal cancer, retinoblastoma, skin cancer, small-cell lung cancer, stomach cancer, testis cancer, tongue cancer, thyroid cancer, uterus cancer, and Wilms' tumor.

Many of the attendant features and advantages of the present disclosure will becomes better understood with reference to the following detailed description considered in connection with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present description will be better understood from the following detailed description read in light of the accompanying drawings, where:

FIG. 1. Synergistic effects of gallates and magnetic force on cell-associated MNPs. Gallic acid (A, B) and methyl gallate (C, D) induced a concentration-dependent increase in the cell-associated MNPs (MNP_{cell}) in LN-229 (A, C) and HeLa (B, D) cells with or without the application of a magnetic field (Mag). Values are means±SE (n=4). *, P<0.05 compared to the corresponding control group. ^{#}, P<0.05 compared to corresponding values in Mag (−).

FIG. 2. Gallates enhanced MNP-cell interaction and internalization. LN-229 cells were incubated with CMX-MNP (100 μg/mL) in the presence of vehicle (PBS; A & C), methyl gallate (30 μM; B & C), or gallic acid (30 μM; C) at 37° C. or 4° C. (C only) with (C only) or without the magnet for 2 h. After incubation, cells were counterstained with LysoTracker (red) for lysosome and DAPI (blue) for nucleus (A &B). *, ^{†}, P<0.05 compared to the corresponding groups of vehicle and gallic acid, respectively. ^{§}, P<0.05 compared to the corresponding groups at 4° C. ^{#}, P<0.05 compared to corresponding values in Mag (−).

FIG. 3. Gallic acid derivatives on MNP_{cell}. LN-229 cells were incubated with MNPs (100 μg/mL) and various gallic acid derivatives at 10 μM (A, C) or 30 μM (B, D) for 24 h in the presence (+) or absence (−) of the magnet (Mag). Values are means±SE (n=4). *, ^{†, §, #}, P<0.05 compared with the corresponding vehicle (v) groups, gallic acid (1), methyl gallate (2), or caffeic acid (9), respectively.

FIG. 4. Polyphenoic compound enhances cellular uptake of MNPs. EGCG (A), ECG (B) and quercetin (C) respectively enhanced cellular uptake of MNPs in LN-299 cells. Data are presented as mean±SE (n=4). *, P<0.05 compared with corresponding values without gallic acid; ^{†}, P<0.05 compared with corresponding values without magnetic field Mag (−).

FIG. 5. Enhanced cellular uptake of MNP (MNP_{cell}) requires simultaneously presence of EGCG and MNPs. Double arrows and dotted area indicate the presence of EGCG and MNP, respectively. Data were presented as mean±SE (n=4). *, P<0.05 compared with corresponding values without EGCG; ^{†}, P<0.05 compared with corresponding values without magnetic field. ^{§}, P<0.05 compared with the corresponding group with EGCG pre-treatment.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### 1. Definitions

For convenience, certain terms employed in the context of the present disclosure are collected here. Unless defined otherwise, all technical and scientific terms used herein have the same meaning as commonly understood by one of the ordinary skill in the art to which this invention belongs.

Unless otherwise indicated, the term “aryl” means an aromatic ring or a partially aromatic ring system composed of carbon and hydrogen atoms. An aryl moiety may comprise multiple rings bound or fused together. Examples of aryl moieties include phenyl, naphthyl, and etc. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is a substituted phenyl.

Unless otherwise indicated, the term “heteroaryl” means an aryl moiety wherein at least one of its carbon atoms has been replaced with a heteroatom (e.g., N, O or S). In some embodiments, a heteroaryl group is a 5-10 membered aromatic or partially aromatic ring system having ring carbon atoms and 1-4 ring heteroatoms provided in the ring system, wherein each heteroatom is independently selected from nitrogen, oxygen, and sulfur (“5-10 membered heteroaryl”). In some examples, the heteroaryl moiety is a substituted benzo[b]tetrahydropyran.

Unless otherwise indicated, the term “substituted,” when used to describe a chemical structure or moiety, refers to a derivative of that structure or moiety wherein one or more of its hydrogen atoms is substituted with an atom, chemical moiety or functional group such as, but not limited to, —OH, —CHO, alkoxy, alkenyl, alkyl (e.g., methyl, ethyl, propyl, t-butyl), aryl, aryloxy, halo, or haloalkyl (e.g., —CCl₃, —CF₃, —C(CF₃)₃).

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

Unless otherwise indicated, the term “magnetic nanoparticle (MNP)” refers to the magnetic nanoparticle composed of a metal core (e.g., magnetite (Fe₃O₄), maghemite (γ-Fe₂O₃), nickel, cobalt, Au and etc) and a polymeric coating designed to increase particle stability/water dispersibility in physiological conditions. A wide variety of therapeutic pharmaceutical agents may be incorporated into the MNP, such as anti-cancer drugs, anti-inflammation drugs, biological response modifiers, corticosteroids and etc. According to preferred embodiments of the present disclosure, each MNP has an anti-cancer drug associated therein. Specific examples of anti-cancer drug include, but are not limited to, methotrexate (MIX), 5-Fluorouracil (5-FU), doxorubicin, epirubicin (FEC), cyclophosphamide, docetaxel, paclitaxel and cisplatin. In addition, the MNP possesses a low-field magnetization when an external magnetic field is applied to the magnetic nanoparticle. Thus, uptake of the present MNP by tumor cells is enhanced when an external field is applied thereto, which leads to higher cytotoxicity or cell killing efficacy towards the tumor cells.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. Pharmaceutically acceptable salts of the compounds of this invention include those derived from suitable inorganic and organic acids and bases. Also included herein are quaternary ammonium salts such as alkylammonium salts. Pharmaceutically acceptable (i.e., non-toxic, physiologically acceptable) salts are preferred, although other salts are useful, for example, in isolation or purification steps which may be employed during preparation. Salts of the present polyphenolic compound (e.g., the compound of formula (I)) may be formed, for example, by reacting the present polyphenolic compound with an amount of acid or base, such as an equivalent amount, in a medium such as one in which the salt precipitates or in an aqueous medium followed by lyophilization. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecyl sulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, m ethanesulfonates, 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates, undecanoates, and the like. Exemplary basic salts (formed, for example, where the R substituents comprise an acidic moiety such as a carboxyl group) include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines, N-methyl-D-glucamines, N-methyl-D-glucamides, t-butyl amines, and salts with amino acids such as arginine, lysine and the like. The basic nitrogen-containing groups may be quaternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

The term “solvate” refers to forms of the compound that are associated with a solvent, usually by a solvolysis reaction. This physical association may include hydrogen bonding. Conventional solvents include water, methanol, ethanol, acetic acid, dimethyl sulfoxide (DMSO), tetrahydrofuran (THF), diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “subject” or “patient” refers to an animal including the human species that is treatable with the compound of the present invention. The term “subject” or “patient” intended to refer to both the male and female gender unless one gender is specifically indicated. Accordingly, the term “subject” or “patient” comprises any mammal which may benefit from the treatment method of the present disclosure.

The term “administered”, “administering” or “administration” are used interchangeably herein to refer a mode of delivery, including, without limitation, intravenously, intramuscularly, intraperitoneally, intraarterially, subcutaneously, or transdermally administering an agent (e.g., a polyphenolic compound or MNPs) of the present invention.

The term “an effective amount” as used herein refers to an amount effective, at dosages, and for periods of time necessary, to achieve the desired result with respect to the treatment of a disease. For example, in the treatment of a tumor, an agent (i.e., the present polyphenolic compound) that enhances the uptake of an anti-cancer agent (i.e., MNPs having an anti-cancer agent associated therein) which results in an increase in the total amount of MNPs in the tumor thereby decrease, prevents, delays or suppresses or arrests the growth of the tumor, would be effective. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two or more doses in a suitable form to be administered at one, two or more times throughout a designated time period.

The term “a sufficient amount” as used herein refers to an amount suffice at dosages, and for periods of time necessary, to achieve the desired result with respect to enhancing the uptake of a component of a treatment regimen (e.g., a magnetic nanoparticle (MNP) having an anti-cancer drug associated therein) so that its total amount in the treatment site is much higher than that of the control. In preferred examples, a sufficient amount of a polyphenolic compound of the present disclosure (e.g., the compound of formula (I)) is concurrently administered with MNPs to tumor cells for a certain period of time such that the amount of MNPs in the tumor cells has increased at least 2 to 15 folds as compared with that of the control cells (i.e., cells treated with MNPs only).

The term “treatment” as used herein are intended to mean obtaining a desired pharmacological and/or physiologic effect, e.g., inhibiting the growth of a tumor. The effect may be prophylactic in terms of completely or partially preventing a disease or symptom thereof and/or therapeutic in terms of a partial or complete cure for a disease and/or adverse effect attributable to the disease. “Treatment” as used herein includes preventative (e.g., prophylactic), curative or palliative treatment of a disease in a mammal, particularly human; and includes: (1) preventative (e.g., prophylactic), curative or palliative treatment of a disease or condition (e.g., an infection) from occurring in an individual who may be pre-disposed to the disease but has not yet been diagnosed as having it; (2) inhibiting a disease (e.g., by arresting its development); or (3) relieving a disease (e.g., reducing symptoms associated with the disease). According to specific embodiments of the present disclosure, an effective amount of the present polyphenolic compound (e.g., the compound of formula (I)) is administered to a subject suffering from a tumor, so that the susceptibility of the tumor cell to certain anti-cancer drug (e.g., 5-FU) is enhanced, thereby the size of the tumor in the subject is reduced, as compared with that of the un-treated subject, and thereby alleviate or ameliorate one or more symptoms associated with the disease, the severity of one or more symptoms associated with the disease and/or the progression of the disease. In preferred embodiments, an effective amount of the polyphenolic compound of the present disclosure is administered together with a magnetic nanoparticle having an anti-cancer drug associated therein, to a subject suffering from a tumor, so as to alleviate or ameliorate one or more symptoms associated with the tumor, and thereby achieving the purpose of treating tumor.

The term “adjuvant” as used herein refers to an agent that does not produce therapeutic effect by itself but help increasing the uptake of a therapeutic agent to the target site or the treatment site (e.g., a tumor) of a subject. According to preferred embodiments of the present disclosure, a polyphenolic compound of the present disclosure acts as an adjuvant to a medicament (e.g., a magnetic nanoparticle having an anti-cancer drug associated therein) for treating a subject in need thereof.

It should also be noted that if the stereochemistry of a structure or a portion of a structure is not indicated with, for example, bold or dashed lines, the structure or the portion of the structure is to be interpreted as encompassing all stereoisomers of it. Similarly, names of compounds having one or more chiral centers that do not specify the stereochemistry of those centers encompass pure stereoisomers and mixtures thereof. Moreover, any atom shown in a drawing with unsatisfied valences is assumed to be attached to enough hydrogen atoms to satisfy the valences. In addition, chemical bonds depicted with one solid line parallel to one dashed line encompass both single and double (e.g., aromatic) bonds, if valences permit.

The singular forms “a”, “and”, and “the” are used herein to include plural referents unless the context clearly dictates otherwise.

For convenience, certain terms employed in the specification, examples and appended claims are collected here. Unless defined otherwise, all technical and scientific terms used herein have the same meaning as commonly understood by one of the ordinary skill in the art to which this invention belongs.

The singular forms “a”, “and”, and “the” are used herein to include plural referents unless the context clearly dictates otherwise.

### 2. Polyphenolic Compounds Enhance Cellular Uptake of MNP

Aspects of the present disclosure relate to the findings that certain polyphenolic compounds can enhance the uptake of a magnetic nanoparticle, which comprises a therapeutic drug (e.g., an anti-cancer drug) associated therein, into cells; thus these polyphenolic compounds may act as an adjuvant to help increasing the amount of the therapeutic drug accumulated in the cells in need of the treatment (e.g., killing cancerous cells).

In one aspect, the present invention relates to a method for enhancing the uptake of a magnetic nanoparticle (MNP), which has an anti-cancer drug associated therein, to a tumor cell. The method comprises concurrently administering to the tumor cell a sufficient amount of a polyphenolic compound and the MNP, so as to enhance the accumulation of MNP in the tumor cell.

According to optional embodiments of the present disclosure, the method further comprises administering a magnetic field to the tumor cell, so that the total amount of the MNP in the tumor cell is at least 2 to 15 folds higher than that of a control cell, which is treated with the MNP only. Preferably, the total amount of MNP in the tumor cell is at least 2, 3, 4, 5, 6, 7, 8, 9, 10, 11, 12, 13, 14, or 15 folds higher than that of the control cell; more preferably, the total amount of MNP in the tumor cell is at least 4 to 12 fold higher than that of the control cell, such as 4, 5, 6, 7, 8, 9, 10, 11, or 12 folds higher than that of the control cell; most preferably, the total amount of MNP in the tumor cell is at least 5 to 10 folds higher than that of the control cell, such as 5, 6, 7, 8, 9, or 10 folds higher than that of the control cell.

Examples of the polyphenolic compound suitable for use in the present method include, but are not limited to, a compound of formula (I), a pharmaceutically acceptable salt, solvate or ester thereof, flavanone, flavone, flavonol, flavan-3-ol, and a combination thereof.

According to embodiments of the present disclosure, the polyphenolic compound is the compound of formula (I),

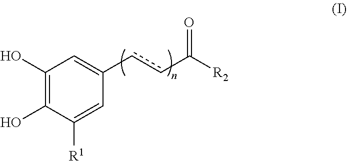

wherein, in the formula (I),

n is 0 or 1;

[symbol] represents a single or double bond;

R₁ is hydrogen or hydroxyl; and

R₂ is hydroxyl, —NH₂, or —OR₃, in which R₃ is heteroaryl optionally substituted with a phenyl having at least two hydroxyl substituents.

Examples of the compound of formula (I) are as follows:

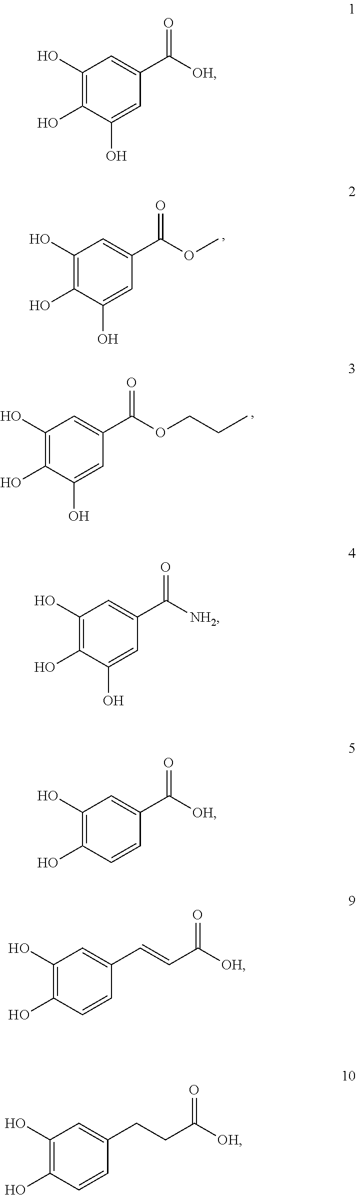

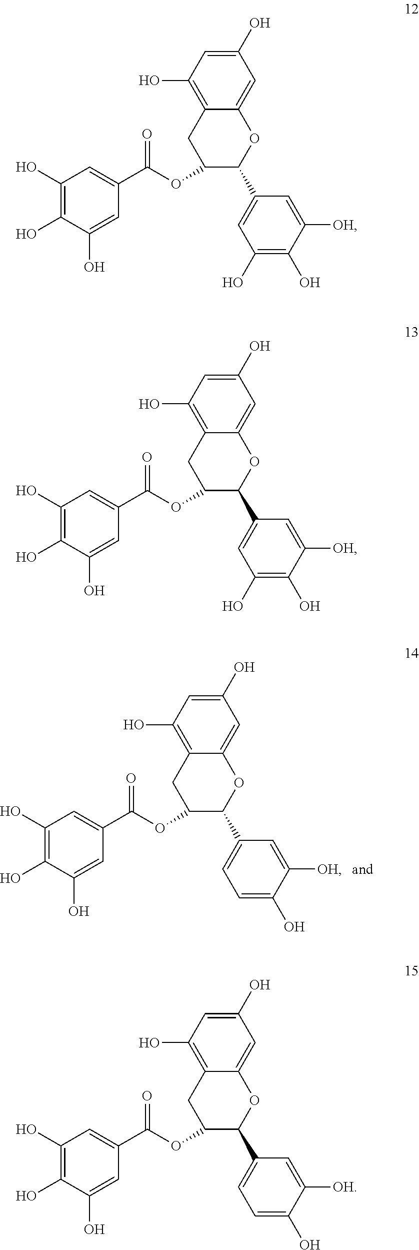

According to some preferred embodiments of the present disclosure, the compound of formula (I) (e.g., compounds 1, 2 and etc) is concurrently administered with MNP to the tumor cell in the presence of a magnetic field, and the total amount of MNP in the tumor cell is at least 2 to 4 folds higher than that of the control cell.

According to still further preferred embodiments of the present disclosure, epigallocatechin gallate (EGCG) is concurrently administered with MNP to the tumor cell in the presence of a magnetic field, and the total amount of MNP in the tumor cell is at least 5 folds higher than that of the control cell.

According to certain embodiments of the present disclosure, flavanone is concurrently administering to the subject with the MNP. Examples of flavanone suitable for use in the present method include, but are not limited to, butin, eruiductyol, and sterubin.

According to some embodiments of the present disclosure, flavaonol is concurrently administering to the subject with the MNP. Examples of flavonol suitable for use in the present method include, but are not limited to, quercetin, fisetin, myricetin, phamnetin, luteoforol, epigallocatechin, and epicatechin. According to certain embodiments of the present disclosure, quercetin is concurrently administering to the subject with the MNP in the presence of a magnetic field, and the amount of MNP in the tumor cell is at least 5 folds higher than of a control cell.

According to certain embodiments of the present disclosure, flavan-3-ol is concurrently administering to the subject with the MNP. Examples of flavan-3-ol suitable for use in the present method include, but are not limited to, epigallocatechin (EGC), catechin, gallocatechin (GC), and epicatechin (EC). According to certain embodiments of the present disclosure, EGC is concurrently administering to the subject with the MNP in the presence of a magnetic field, and the total amount of MNP in the tumor cell is at least 12 folds higher than that of the control cell.

According to certain embodiments of the present disclosure, flavone is concurrently administering to the subject with the MNP. Examples of flavone suitable for use in the present method include, but are not limited to, luteolin, 6-hydroxyluteolin, tricetin, hypolaetin, and nepetin.

### 3. Method of Treatment

In a further aspect, the present disclosure is directed to a method for treating a cancer of a subject. The method comprises concurrently administering to the subject an effective amount of a polyphenolic compound described above and a magnetic nanoparticle (MNP) having an anti-cancer drug associated therein, so as to ameliorate or alleviate symptoms associated with the cancer.

Exemplary polyphenolic compound suitable for concurrently administered with MNP to the subject may be any of a compound of formula (I), a pharmaceutically acceptable salt, solvate or ester thereof, flavanone, flavone, flavonol, flavan-3-ol, or a combination thereof. The compound of formula (I) has the structure of:

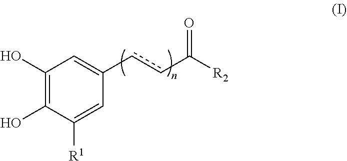

wherein, in the formula (I),

n is 0 or 1;

[symbol] represents a single or double bond;

R₁ is hydrogen or hydroxyl; and

R₂ is hydroxyl, —NH₂, or —OR₃, in which R₃ is heteroaryl optionally substituted with a phenyl having at least two hydroxyl substituents.

Additionally or optionally, the method further comprises administering a magnetic field to the cancer.

According to preferred embodiments of the present disclosure, the compound of formula (I) is concurrently administering to the subject with the MNP, wherein in the formula (I), n is 0, and R₁ and R₂ are respectively hydroxyl.

According to certain embodiments of the present disclosure, the compound of formula (I) is concurrently administering to the subject with the MNP, wherein in the formula (I), n is 0, R₁ is hydroxyl, and R₂ is methoxyl.

According to certain embodiments of the present disclosure, the compound of formula (I) is concurrently administering to the subject with the MNP, wherein in the formula (I), n is 0, R₁ is hydroxyl, and R₂ is propoxyl.

According to certain embodiments of the present disclosure, the compound of formula (I) is concurrently administering to the subject with the MNP, wherein in the formula (I), n is 0, R₁ is hydroxyl, and R₂ is —NH₂.

According to certain embodiments of the present disclosure, the compound of formula (I) is concurrently administering to the subject with the MNP, wherein in the formula (I), n is 0, R₁ is hydrogen, and R₂ is hydroxyl.

According to certain embodiments of the present disclosure, the compound of formula (I) is concurrently administering to the subject with the MNP, wherein in the formula (I), n is 1, [symbol] is a double bond, R₁ is hydrogen, and R₂ is hydroxyl.

According to certain embodiments of the present disclosure, the compound of formula (I) is concurrently administering to the subject with the MNP, wherein in the formula (I), n is 1, [symbol] is a single bond, R₁ is hydrogen, and R₂ is hydroxyl.

According to certain embodiments of the present disclosure, the compound of formula (I) is concurrently administering to the subject with the MNP, wherein in the formula (I), n is 0, R₁ is hydroxyl, and R₂ is

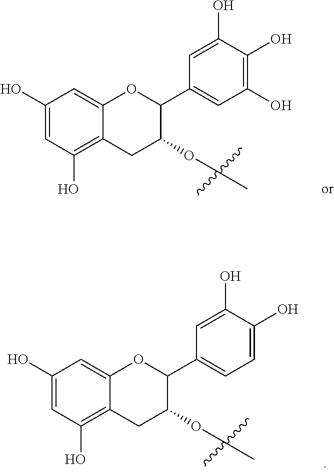 [SMILES: *O[C@@H]1Cc2c(O)cc(O)cc2OC1c1cc(O)c(O)c(O)c1 ; *O[C@@H]1Cc2c(O)cc(O)cc2OC1c1ccc(O)c(O)c1]

According to certain embodiments of the present disclosure, flavanone is concurrently administering to the subject with the MNP. Examples of flavanone suitable for use in the present method include, but are not limited to, butin, eruiductyol, and sterubin.

According to certain embodiments of the present disclosure, flavaonol is concurrently administering to the subject with the MNP. Examples of flavonol suitable for use in the present method include, but are not limited to, quercetin, fisetin, myricetin, phamnetin, luteoforol, epigallocatechin, and epicatechin. According to certain embodiments of the present disclosure, quercetin is concurrently administering to the subject with the MNP.

According to certain embodiments of the present disclosure, flavan-3-ol is concurrently administering to the subject with the MNP. Examples of flavan-3-ol suitable for use in the present method include, but are not limited to, epigallocatechin (EGC), catechin, gallocatechin (GC), and epicatechin (EC). According to certain embodiments of the present disclosure, EGC is concurrently administering to the subject with the MNP.

According to certain embodiments of the present disclosure, flavone is concurrently administering to the subject with the MNP. Examples of flavone suitable for use in the present method include, but are not limited to, luteolin, 6-hydroxyluteolin, tricetin, hypolaetin, and nepetin.

Examples of cancer that may be treated by the present method include, but are not limited to, bladder cancer, bone cancer, bone marrow cancer, brain cancer, breast cancer, cholangiocarcinoma, colon cancer, esophagus cancer, Ewing's sarcoma, gastro-intestine cancer, gum cancer, head cancer, Hodgkin's disease, kidney cancer, liver cancer, lung cancer, larynx cancer, melanoma, multiple myeloma, nasopharynx carcinoma, non-small-cell lung (NSCL) cancer, leukemia, liver cancer, nasopharynx cancer, neck cancer, neuroblastoma, ovary cancer, pancreatic cancer, prostate cancer, rectal cancer, retinoblastoma, skin cancer, small-cell lung cancer, stomach cancer, testis cancer, tongue cancer, thyroid cancer, uterus cancer, and Wilms' tumor.

The present invention will now be described more specifically with reference to the following embodiments, which are provided for the purpose of demonstration rather than limitation. While they are typically of those that might be used, other procedures, methodologies, or techniques known to those skilled in the art may alternatively be used.

### EXAMPLES

Materials and Methods

Materials.

Dextran-coated magnetic nanoparticles (MNPs, Nanomag®-D COOH; 250 nm) were purchased from micromod Partikel-technologie GmbH (Rostock, Germany); green fluorescent carboxymethyl-dextran coated MNPs (nano-screenMAG-CMX; 200 nm) was purchased from Chemicell GmbH (Berlin, Germany). Dulbecco's modified Eagle's medium (DMEM), minimum essential media (MEM), and trypsin-EDTA were purchased from Gibco BRL (Grand Island, N.Y.). Penicillin/streptomycin/amphotericin B was purchased from Upstate (Lake Placid, N.Y.). Fetal bovine serum (FBS), ammonium persulfate, potassium thiocyanate, caffeic acid (i.e., compound 9), ferulic acid (i.e., compound 11), Epigallocatechin gallate (EGCG, i.e., compound 12), Epicatechin gallate (ECG, i.e., compound 13), gallocatechin gallate (GCG, i.e., compound 14), catechin gallate (CG, i.e., compound 15), hydrochloric acid, paraformaldehyde, 2,2-diphenyl-1-picrylhydrazyl (DPPH), and Cell Counting Kit-8 (CCK-8) were purchased from Sigma-Aldrich (St. Louis, Mo.). Lysotracker® and 4′,6-diamidino-2-phenylindole (DAPI) were purchased from Invitrogen (Carlsbad, Calif.). Trimethoxybenzoic acid (i.e., compound 8), protocatechuic acid (i.e., compound 5), and α-resorcylic acid (i.e., compound 7) were purchased from TCI (Tokyo, Japan). Gallic acid monohydrate (i.e., compound 1) was from Janssen Chimica (Beerse, Belgium); gallamide (3,4,5-trihydroxybenzamide, compound 4)) was from Alfa Aesar (Heysham, Lancashire, UK); p-hydroxybenzoic acid (i.e., compound 6) was purchased from Merck (Darmstadt, Germany).

Cell Culture.

LN229 cells, human glioma cells, were cultured in DMEM medium; whereas HeLa cells, human cervical cancer cells, were cultured in MEM medium. Both media were supplemented with 10% fetal bovine serum (FBS) and 1% penicillin/streptomycin/amphotericin B solution. The cells were maintained in a 37° C. incubator supplied with 5% CO₂ and sub-cultured every 3 to 4 days.

Determination of Cell-Associated MNPs.

LN229 or HeLa cells were seeded and grown in 24-well plates. After attaining 80-90% confluence, the cells were exposed to MNPs (100 μg/mL) and phenolic compounds (10 or 30 μM) in the presence or absence of NdFeB magnet underneath the plate for 24 h. In some experiments, cells were equilibrated at 4° C. for 20 min, followed by an additional 2-h incubation with MNPs at 4° C. in the absence and presence of the magnet. A home-made magnetic plate with 24 pieces of cylindrical NdFeB magnet were used to provide a magnetic field of 3.4 kG at the center of each well. For the group without the magnet during incubation with MNPs, the magnetic plate was placed underneath the culture plates for 5 min after MNP administration to facilitate MNP sedimentation. The cells were then washed twice with phosphate-buffered saline (PBS) and trypsinized prior to colorimetric quantification. The cell pellet containing cell-associated MNPs (MNP_{cell}) was treated with 10% hydrochloric acid at 55° C. for 4 h, followed by adding ammonium persulfate (1 mg/mL) and potassium thiocyanate solution (1 M). The amount of cell-associated iron was determined with VICTOR3 Multilabel Plate Reader (PerkinElmer, Waltham, Mass.) at OD₄₉₀. A calibration curve was prepared under identical conditions.

Confocal Microscopy.

LN229 cells were seeded onto poly-lysine coated coverslips 18 h before experiments. The cells were incubated with green fluorescent MNPs with carboxylmethyl-dextran coating (nano-screenMAG-CMX; 100 μg/mL) and methyl gallate (30 μM) at 37° C. for 2 h in the presence of NdFeB magnet underneath the culture plate. Cells were then washed twice with PBS and then incubated with Lysotracker® (0.125 μM) for 30 min for staining of lysosomes. The cells were than washed with PBS and fixed with 4% paraformaldehyde for 20 min at room temperature. The cells were counterstained with DAPI and imaged with a Zeiss LSM 510 Meta laser confocal microscope system equipped with a 100×/1.4 oil immersion objective lens. Image processing and analysis of fluorescent MNPs colocalized with cells was performed using Fiji Image J.

Cellular Toxicity Assay.

The cytotoxicity of the compound of formula (I) for LN-229 cells were measured using a CCK-8 kit according to manufacturer's instruction. Briefly, LN-229 cells were cultured in a 24-well plate to 80-90% confluence before incubation with the designated compound (10 or 30 μM) or in combination with MNP (100 μg/mL). After administration of MNPs, a magnet was placed underneath for 5 min or 2 h. Then, cells were washed with PBS and incubated with medium containing 10% of CCK-8 solution for additional one hour. The absorbance of each sample at 450 nm was determined with a microplate reader (VICTOR3 Multilabel Plate Reader, PerkinElmer, Waltham, Mass.). The percentage of viability was calculated as following: (viable cells) %=(OD of drug-treated sample/OD of untreated sample)×100.

Statistical Analysis.

Results are expressed as mean±SE. Statistical evaluation of the data was performed with Student's t-test for simple comparisons between two values when appropriate. For multiple comparisons, results were analyzed by 2-way analysis of variance (ANOVA) followed by Duncan's post-hoc test. A value of P<0.05 was considered statistically significant.

### Example 1 Preparation of the Compound of Formula (I)

In general, the compound of formula (I) of the present disclosure, particularly, compounds 1, 4, 5, 9, and 11-15 were obtained from commercial sources as described in the “Materials and Methods” section. Compounds 2, 3, and 10 were synthesized in accordance with procedures described bellowed.

1.1 Methyl Gallate (Compound 2)

To a stirred suspension of gallic acid monohydrate (4.09 g, 21.7 mmol) in methanol (16 mL) at 0° C. was added dropwisely thionyl chloride (2 mL, 27.6 mmol). The mixture was heated under reflux for 3.5 h. The reaction mixture was then poured into crushed ice. The resulted precipitate was filtered and washed thoroughly with water. The crude product was recrystallized from n-hexane-ethyl acetate (EA) to provide methyl gallate as white crystal, mp 199-201° C.

¹H NMR (300 MHz, CD₃OD) δ (ppm) 3.82 (s, 3H), 7.04 (s, 2H); ¹³C NMR (125 MHz, CD₃OD) δ (ppm) 52.4, 110.2, 121.6, 139.9, 146.7, 169.2.

1.2 Propyl Gallate (Compound 3)

To a stirred suspension of gallic acid monohydrate (0.30 g, 1.6 mmol) in 1-propanol (5 mL), thionyl chloride (0.8 mL, 11 mmol) was added dropwisely. The mixture was heated under reflux for 5.5 hr. The reaction mixture was poured into crushed ice and extracted with 70 mL of ethyl acetate for three times. The combined organic layer was washed with water for several times, dried over MgSO₄, and evaporated under reduced pressure. The crude product was recrystallized from aqueous ethanol to provide propyl gallate as white crystal, mp 145-148° C.

¹H NMR (300 MHz, CD₃OD) δ (ppm) 1.03 (t, J=7.5 Hz, 3H), 1.76 (m, 2H), 4.18 (t, J=6.5 Hz, 2H), 7.05 (s, 2H) [39]; ¹³C NMR (125 MHz, CD₃OD) δ (ppm) 11.0, 23.4, 67.4, 110.2, 121.9, 139.9, 146.6, 168.8.

1.3 Dihydrocaffeic Acid (Compound 10)

In a hydrogenation bottle placed 51.5 mg of 5% Pd/C and a solution of caffeic acid (0.5047 g, 2.8 mmol) in methanol (16 mL). The hydrogenation was performed in a Parr hydrogenation apparatus at 26-35 psi pressure for 3.5 h. The Pd/C was filtered off through a pad of celite and the filtrate was evaporated under reduced pressure. The crude product was recrystallized from water to provide dihydrocaffeic acid as an off-white crystal, mp 138.5-140° C.

¹H NMR (300 MHz, CD₃OD) δ (ppm) 2.51 (t, J=7.8 Hz, 2H), 2.76 (t, J=7.8 Hz, 2H), 6.52 (dd, J=7.8, 2.2 Hz, 1H), 6.65 (d, J=2.2 Hz, 1H), 6.66 (d, J=7.8 Hz, 1H); ¹³C NMR (125 MHz, CD₃OD) δ (ppm) 31.7, 37.4, 116.5, 116.6, 120.6, 133.9, 144.7, 146.3, 177.2.

### Example 2 Characterization of the Present Polyphenolic Compound on Cellular Uptake of Magnetic Nanoparticles (MNPs)

2.1 the Compound of Formula (I) Increases Uptake of MNPs in Tumor Cells

2.1.1 Compounds 1 and 2 Respectively Increased the Uptake of MNPs

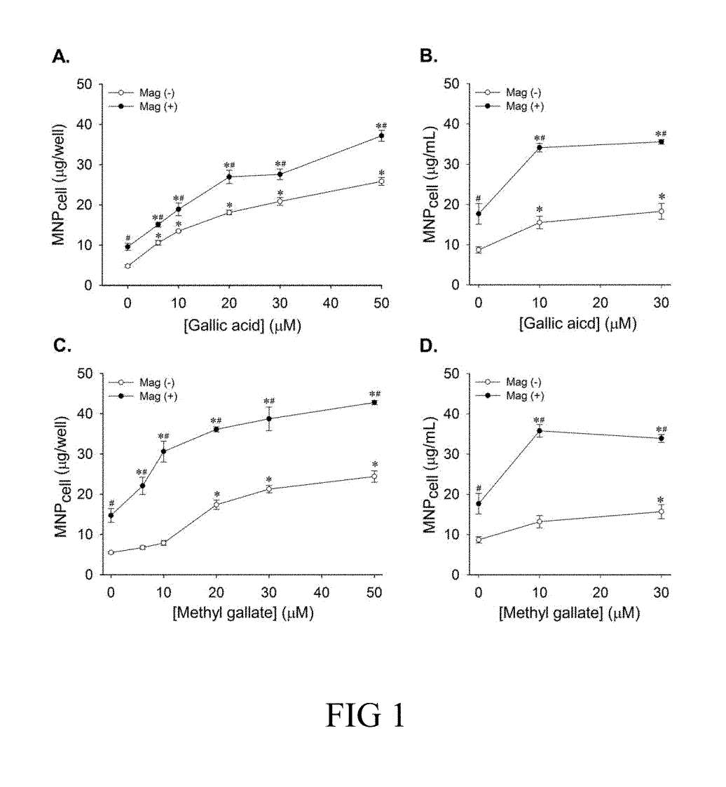

References is first made to FIG. 1, which are line graphs respectively illustrate the effects of gallic acid (i.e., compound 1) and methyl gallate (i.e., compound 2) on the uptake of MNPs in LN299 cells (FIG. 1, panels A and C) and Hela cells (FIG. 1, panels B and D).

Compared to the vehicle group, gallic acid (i.e., compound 1) at concentration as low as 6 μM induced a significant increase in LN229 cell-associated MNPs with and without the application of a magnet; at 50 μM, gallic acid increased MNP_{cell} by 5.4- and 3.9-fold in the absence and presence of the magnet, respectively (FIG. 1, panel A). Similar effects were observed in HeLa cells, where gallic acid at 10 μM increased MNP_{cell} by 1.8- and 1.9-fold in the absence and presence of the magnet, respectively (FIG. 1, panel B).

Methyl gallate (i.e., compound 2), a methyl ester of gallic acid, also enhanced MNP_{cell} in a concentration-dependent manner with or without magnet (FIG. 1, panel C). In LN229 cells, methyl gallate increased MNP_{cell} by 1.4-fold at 10 μM and further increased MNP_{cell} by 4.5-fold at 50 μM in the absence of the magnet (FIG. 1, panel C). With the application of the magnet underneath LN229 cells, methyl gallate increased MNP_{cell} by 2.1-fold at 10 μM and 2.9-fold at 50 μM, respectively (FIG. 1, panel C). Similar synergetic effects were observed in HeLa cells, where methyl gallate at 10 μM induced a 0.6- vs. 1.0-fold increase of MNP_{cell} in the absence and presence of the magnet (FIG. 1, panel D)

2.1.2 Compound 2 Enhanced the Internalization of MNPs

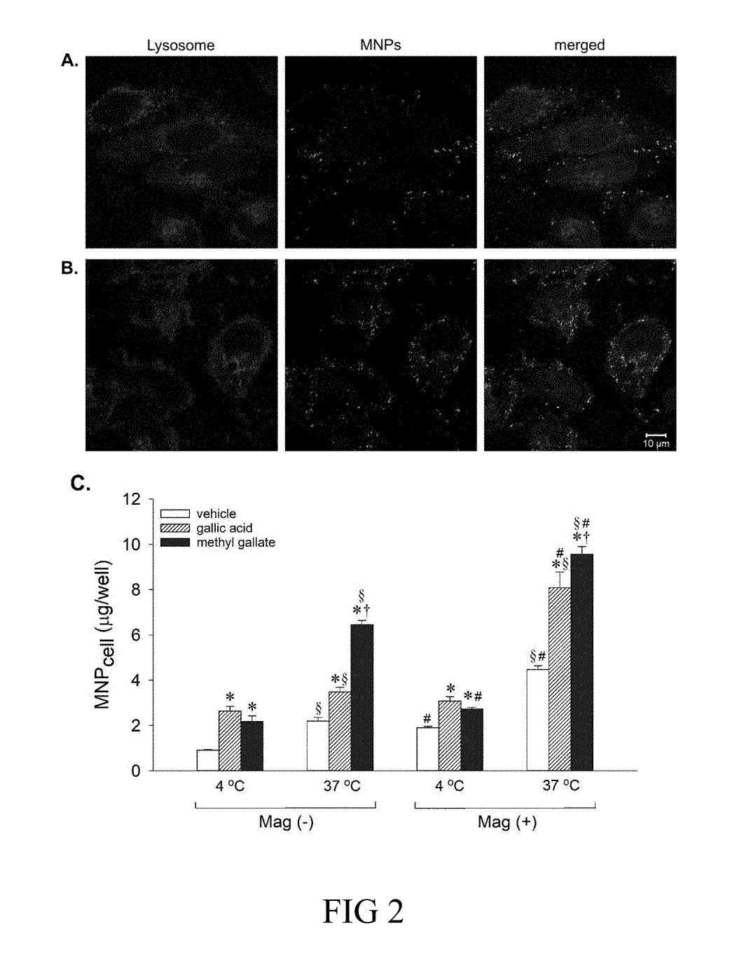

To characterize the underlying mechanism for the present compound induced cellular uptake of MNP, green fluorescent CMX-MNPs were synthesized and incubated with LN229 cells in the presence of methyl gallate (i.e., compound 2). Results are provided in FIG. 2.

It was found that methyl gallate (30 μM; 2 h) greatly increased internalization of MNPs (FIG. 2, panel B), as compared to that of the vehicle control (FIG. 2, panel A). Further, as the green fluorescent signals distributed outside the nucleus appeared to be blue, suggesting that MNPs were internalized into the cytoplasm, but not into the nucleus. In addition, co-localization of internalized MNPs with lysosomes was also observed in the cytoplasm (FIG. 2, panel B), suggesting that MNP internalization was predominantly mediated by endocytosis. The overlap coefficient, Mander's coefficient, of MNP relative to lysosomes in the absence and presence of methyl gallate were 0.51±0.08 (n=6) and 0.65±0.03 (n=8) respectively, suggesting that methyl gallate may increase MNP internalization.

Further, it was found that incubating gallates and MNPs with LN229 cells at 4° C. significantly reduced MNP_{cell} in all groups studied, suggesting the endocytosis of MNPs was suppressed at low temperature (FIG. 2, panel C). Compared with the MNPs uptake at 37° C., MNP uptake at 4° C. was reduced to 42% or 43% in the absence or presence of magnet, respectively. At 4° C., gallic acid and methyl gallate independently enhanced MNP_{cell} by 1.9- and 1.4-folds without the application of magnet, and 0.6- or 0.4-fold with the magnet, respectively.

2.1.3 Gallic Acid Derivatives Increased the Uptake of MNPs

In this example, gallic acid (compound 1) and its derivatives (compounds 2-15) were either purchased from commercial sources or synthesized in according to procedures described in Example 1, and then their effects on cellular uptake of MNPs were investigated. Respective structures of compounds 1 to 15 are as illustrated bellow:

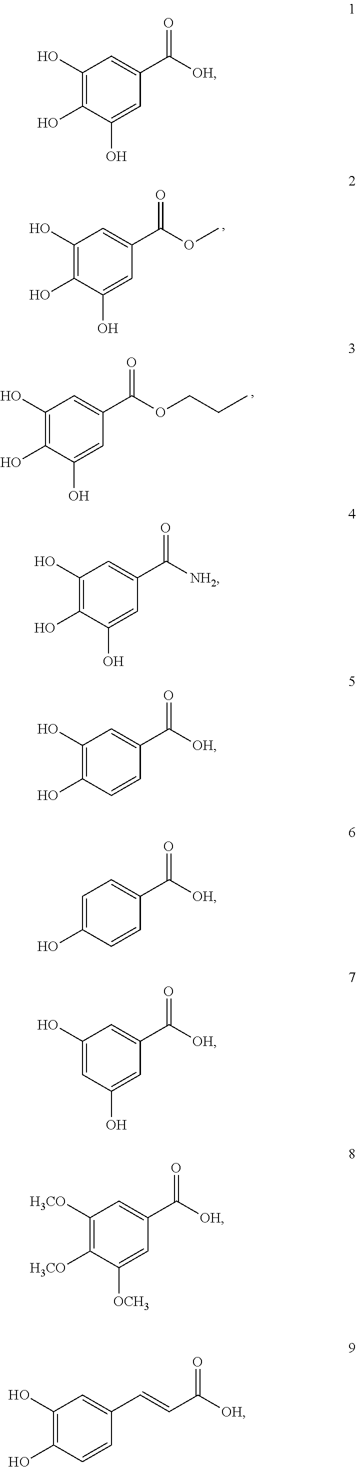

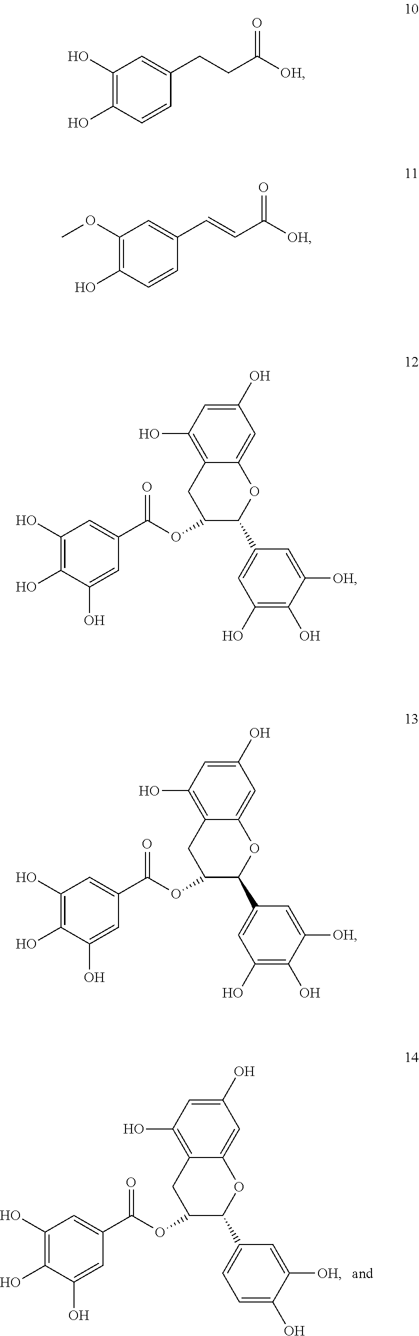

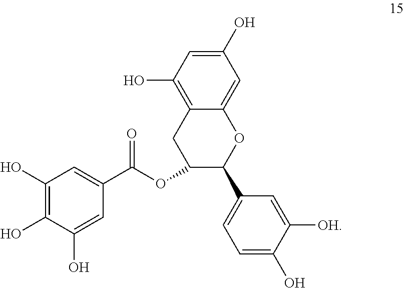

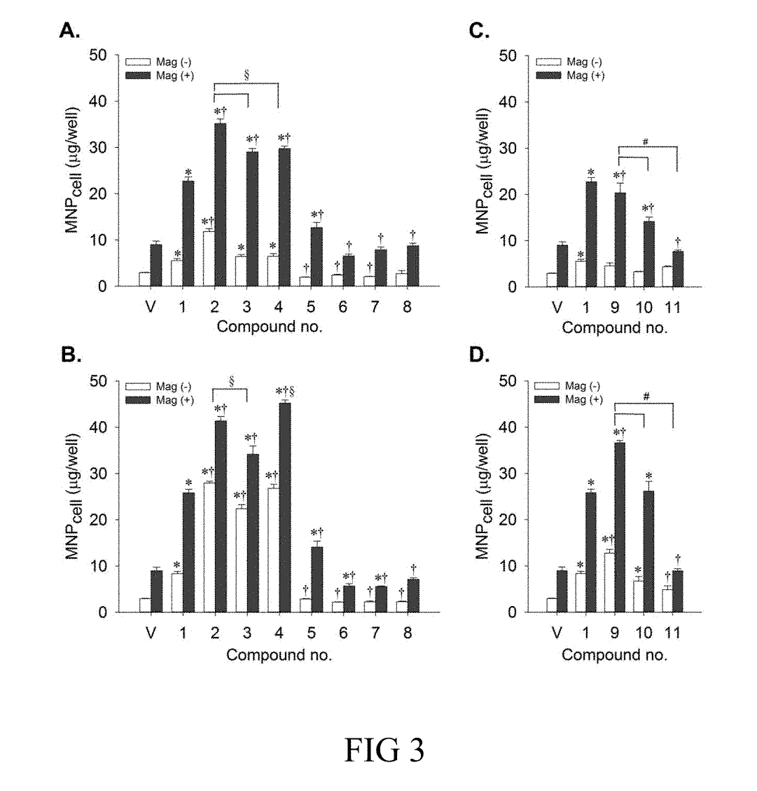

Reference is made to FIG. 3, in which LN-299 cells were incubated with MNPs (100 μg/mL), and any of the compounds 1-11 (at the concentration of 10 or 30 μM) for 24 hrs, then MNP_{cell} in the presence or absence of magnet was determined. It was found that, gallic acid (compound 1) and some of its derivatives (i.e., compounds 2, 3, and 4) were all effective in enhancing the uptake of MNPs, as compared to that of the vehicle control; and the increase in MNPs uptake was more significant in the presence of magnet. As to compounds 5, 9 and 10, they independently enhanced the uptake of MNPs in the presence of magnet, but not in the absence of magnet. As to the compounds 6, 7, 8 and 11, none of them was effective in increasing the amount of MNP in the tumor cells, even with the application of magnet.

2.2 Epigallocatechin-3-Gallate (EGCG), Epicatechin Gallate (ECG) and Quercetin Independently Increased the Uptake of MNPs in Tumor Cells

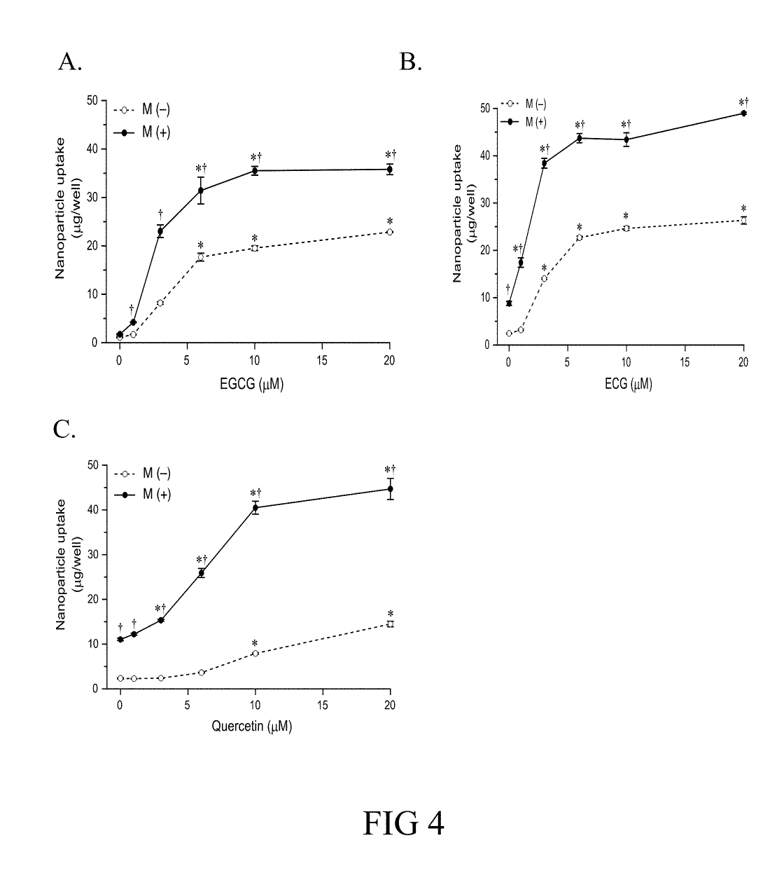

In this example, epigallocatechin-3-gallate (EGCG, or compound 12), epicatechin gallate (ECG, or compound 14) and quercetin were also tested for their capabilities in enhancing cellular uptake of MNPs. Results are illustrated in FIG. 4.

As the data in FIG. 4 depicted, epigallocatechin-3-gallate (EGCG), epicatechin gallate (ECG) and quercetin, all possessed the capability of enhancing cellular uptake of MNPs. The cellular uptake of MNPs was significantly increase by EGCG at the concentration as low as 3 μM. At 10 μM, EGCG increased cellular uptake of MNP by 5.7 times in a magnetic field-free environment and by 16 times with the presence of an external magnetic field, in comparison with the cases without EGCG (FIG. 4, panel A). Similarly to EGCG, the cellular uptake of MNP was also significantly increase by ECG as low as 3 μM. At 10 μM, ECG can increase cellular uptake of MNP by 12 times in a magnetic field-free environment and by 5-6 times with an external magnetic field, in comparison with the cases without ECG (FIG. 4, panel B). As to quercetin, in the absence of magnetic field, the cellular uptake of MNP in the presence of quercetin (20 μM) was 5 times higher than that without quercetin. In the presence of magnet, quercetin exerted a more significant increase in the uptake of MNP via a concentration-dependent manner.

### Example 3 EGCG Enhanced Cellular Uptake of MNPs Required Simultaneous Presence of EGCG and MNPs

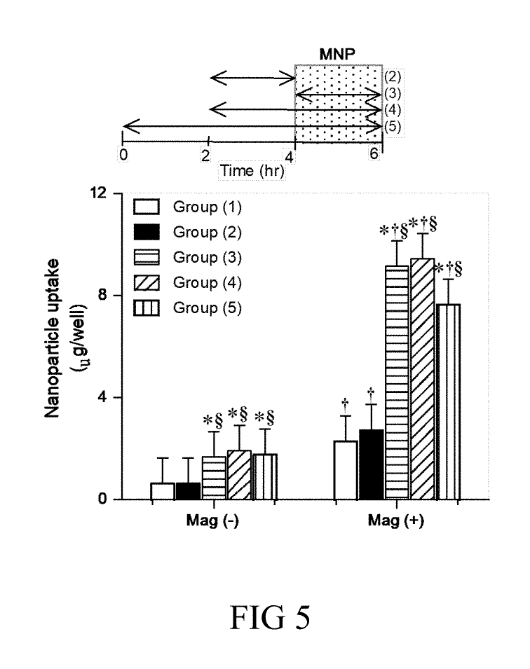

In this example, the timing for adding the present polyphenolic compound with MNPs to the tumor cells was investigated. To this purpose, 5 study groups were designed and respectively treated with the designated compounds, then the MNP_{cell} in each group was determined. The concentrations of MNPs and epigallocatechin gallate (EGCG) or compound 12 used in this example were respectively 100 μg/mL and 10 μM. In Group I (the control group), cells were treated with MNPs (2 hrs). In Group 2, cells were first treated with EGCG (2 hrs), which was removed, and then with MNPs (2 hrs). In Group 3, cells were simultaneously treated with EGCG and MNPs for 2 hours. In Group 4, cells were first treated with EGCG (2 hrs), then with the combination of MNP and EGCG (2 hrs). In Group 5, cells were first treated with EGCG (4 hrs), then, with the combination of MNP and EGCG (2 hrs). Results are depicted in FIG. 5.

Comparing the data of Groups 2 and 3, it is evident that when EGCG and MNPs were incubated with cells independently, then no enhanced uptake of MNPs was found (Group 2); by contrast, when EGCG and MNPs were simultaneously present in the culture media, then enhanced uptake of MNPs was achieved, both in the presence and absence of magnet. Further, pre-treating the cells with EGCG (for 2 or 4 hrs) did not result in further increase in the cellular uptake of MNPs, in which MNPs were added simultaneously with EGCG to cells (Group 3 vs Groups 4 and 5).

Taken together, the data in this example indicated that for enhanced cellular uptake of MNPs, EGCG must be present simultaneously with MNPs to achieve such effect.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method for enhancing the uptake of a magnetic nanoparticle having an anti-cancer drug associated therein to a tumor cell, comprising:
concurrently administering to the tumor cell a sufficient amount of a polyphenolic compound of formula (I), a pharmaceutically acceptable salt, solvate or ester thereof, and the magnetic nanoparticle, 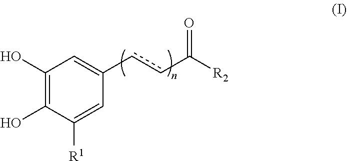
wherein, in the formula (I),
n is 0 or 1; [symbol]
represents a single or double bond;
R₁ is hydrogen or hydroxyl; and
R₂ is hydroxy, —NH₂, or —OR₃, in which R₃ is a heteroaryl optionally substituted with a phenyl having at least two hydroxyl substituents.

2. The method of claim 1, further comprising administering a magnetic field to the tumor cell.

3. The method of claim 2, wherein n is 0, and R₁ and R₂ are respectively hydroxyl.

4. The method of claim 2, wherein n is 0, R₁ is hydroxyl, and R₂ is methoxyl.

5. The method of claim 2, wherein n is 0, R₁ is hydroxyl, and R₂ is propoxyl.

6. The method of claim 2, wherein n is 0, R₁ is hydroxyl, and R₂ is —NH₂.

7. The method of claim 2, wherein n is 0, R₁ is hydrogen, and R₂ is hydroxyl.

8. The method of claim 2, wherein n is 1, [symbol] is a double bond, R₁ is hydrogen, and R₂ is hydroxyl.

9. The method of claim 2, wherein n is 1, [symbol] is a single bond, and R₁ is hydrogen, and R₂ is hydroxyl.

10. The method of claim 2, wherein n is 0, R₁ is hydroxyl, and R₂ is

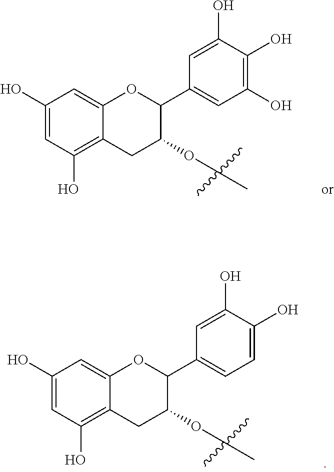 [SMILES: *O[C@@H]1Cc2c(O)cc(O)cc2OC1c1cc(O)c(O)c(O)c1 ; *O[C@@H]1Cc2c(O)cc(O)cc2OC1c1ccc(O)c(O)c1]

11. A method for enhancing the uptake of a magnetic nanoparticle having an anti-cancer drug associated therein to a tumor cell comprising:
concurrently administering to the tumor cell a sufficient amount of a polyphenolic compound and the magnetic nanoparticle, wherein the polyphenolic compound is selected from the group consisting of flavanone, flavone, flavonol, and flavan-3-ol.

12. The method of claim 10, further comprising administering a magnetic field to the tumor cell.

13. The method of claim 11, wherein the flavanone is butin, eruiductyol, or sterubin.

14. The method of claim 11, wherein the flavonol is quercetin, fisetin, myricetin, phamnetin, luteoforol, epigallocatechin, or epicatechin.

15. The method of claim 11, wherein the flavan-3-ol is epigallocatechin (EGC), catechin, gallocatechin (GC), or epicatechin (EC).

16. The method of claim 11, wherein the flavone is luteolin, 6-hydroxyluteolin, tricetin, hypolaetin, or nepetin.

17. A method of treating a cancer of a subject comprising:
concurrently administering to the subject an effective amount of a polyphenolic compound and a magnetic nanoparticle having an anti-cancer drug associated therein, so as to ameliorate or alleviate symptoms associated with the cancer, wherein,
the polyphenolic compound is selected from the group consisting of a compound of formula (I), a pharmaceutically acceptable salt, solvate or ester thereof, flavanone, flavone, flavonol, and flavan-3-ol; 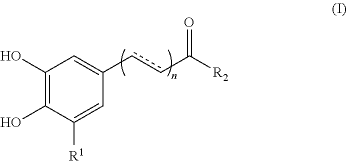
wherein, in the formula (I),
n is 0 or 1; [symbol]
represents a single or double bond;
R₁ is H or hydroxyl; and
R₂ is hydroxyl, —NH₂, or —OR₃, in which R₃ is a heteroaryl optionally substituted with a phenyl having at least two hydroxyl substituents.

18. The method of claim 17, further comprising administering a magnetic field to the subject.

19. The method of claim 18, wherein the compound of formula (I) is selected from the group consisting of,

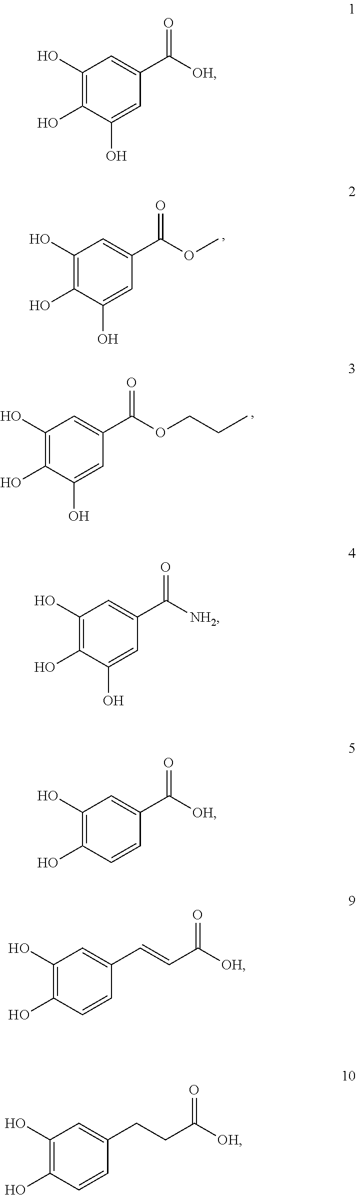

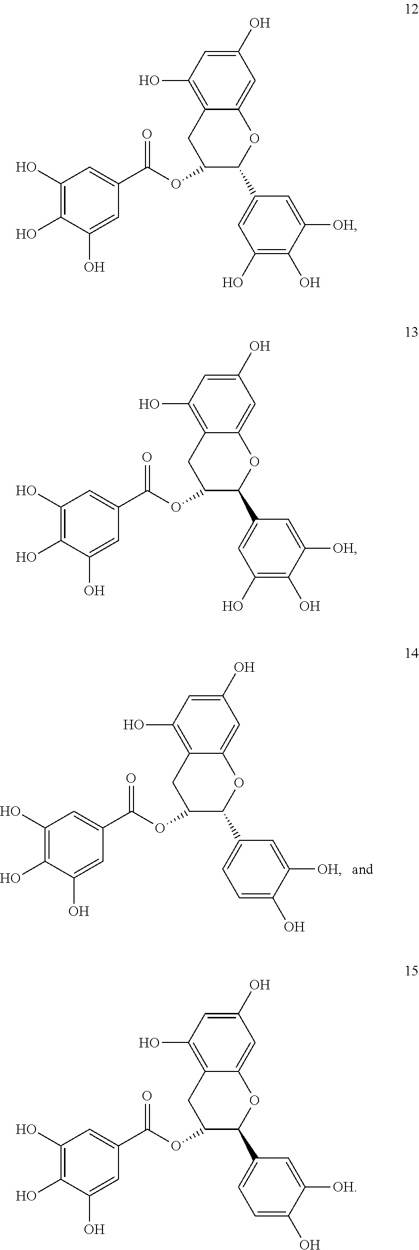

20. The method of claim 18, wherein the flavanone is butin, eruiductyol, or sterubin.

21. The method of claim 18, wherein the flavonol is quercetin, fisetin, myricetin, phamnetin, luteoforol, epigallocatechin, or epicatechin.

22. The method of claim 18, wherein the flavan-3-ol is epigallocatechin (EGC), catechin, gallocatechin (GC), or epicatechin (EC).

23. The method of claim 18, wherein the flavone is luteolin, 6-hydroxyluteolin, tricetin, hypolaetin, or nepetin.

24. The method of claim 18, wherein the cancer is selected from the group consisting of bladder cancer, bone cancer, bone marrow cancer, brain cancer, breast cancer, cholangiocarcinoma, colon cancer, esophagus cancer, Ewing's sarcoma, gastro-intestine cancer, gum cancer, head cancer, Hodgkin's disease, kidney cancer, liver cancer, lung cancer, larynx cancer, melanoma, multiple myeloma, nasopharynx carcinoma, non-small-cell lung (NSCL) cancer, leukemia, liver cancer, nasopharynx cancer, neck cancer, neuroblastoma, ovary cancer, pancreatic cancer, prostate cancer, rectal cancer, retinoblastoma, skin cancer, small-cell lung cancer, stomach cancer, testis cancer, tongue cancer, thyroid cancer, uterus cancer, and Wilms' tumor.
